(19) United States
(12) Patent Application Publication
(10) Pub. No.: US 2007/0219154 A1
(43) Pub. Date: Sep. 20, 2007

(54) Methods for preventing and/or treating a cell proliferative disorder

(76) Inventors: Suxing Liu, Edison, NJ (US); Jean E. Lachowicz, Berkeley Heights, NJ (US); Ho-Sam Ahn, Montville, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/642,487

(22) Filed: Dec. 20, 2006

Related U.S. Application Data
(60) Provisional application No. 60/752,153, filed on Dec. 20, 2005.

(51) Int. Cl. A61K 48/00; A61K 31/444; A61K 31/4433; A61K 38/21

(52) U.S. Cl. 514/44; 514/337; 514/332; 424/85.7

(57) ABSTRACT

The present invention relates to methods for preventing and/or treating the growth and/or metastasis of a cell proliferative disorder. In particular, the methods include use of a protease activated receptor-1 (PAR-1) inhibitor.

### REFERENCE TO RELATED APPLICATION

This application claims the benefit of priority to U.S. Provisional Patent Application 60/752,153 filed Dec. 20, 2005, the entire disclosure of the priority application is hereby incorporated by reference.

### FIELD OF THE INVENTION

The present invention relates to methods for preventing and/or treating the growth and/or metastasis of a cell proliferative disorder. In particular, the methods include use of a protease activated receptor-1 (PAR-1) inhibitor.

### BACKGROUND OF THE INVENTION

For the year 2005, the American Cancer Society estimates the number of new cancer cases at 1,372,910 and the number of cancer related deaths at 570,280 in the United States alone. In light of the widespread number of cancer cases and cancer-related deaths, as well as the inadequacies of currently available treatments, there is a need for more effective methods to prevent and/or treat cancer. Such cancers include carcinoma (including breast cancer, prostate cancer, lung cancer, colorectal and/or colon cancer, hepatocellular carcinoma, melanoma), lymphoma (including non-Hodgkin's lymphoma and mycosis fungoides), leukemia, sarcoma, mesothelioma, brain cancer (including glioma), germinoma (including testicular cancer and ovarian cancer), choriocarcinoma, renal cancer, pancreatic cancer, thyroid cancer, head and neck cancer, endometrial cancer, cervical cancer, bladder cancer, or stomach cancer.

A crucial step in continuous growth of tumors and development of metastasis is the recruitment of new blood vessels in and around tumors. A tumor mass <1 mm in diameter can receive oxygen and nutrients by diffusion, but any increase in tumor mass requires angiogenesis, i.e., the proliferation and morphogenesis of vascular endothelial cells. Despite advances in the prevention and/or treatment of cancer, there remains a need for more effective methods to prevent and/or treat the growth and metastasis of a variety of cancers, including pancreatic cancer.

### SUMMARY OF THE INVENTION

The present invention provides methods useful for preventing and/or treating the growth and/or metastasis of a cell proliferative disorder in a patient disposed to or suffering therefrom comprising administering a therapeutically effective amount of a protease activated receptor-1 (PAR-1) inhibitor.

In one embodiment, the cell proliferative disorder is renal cancer, hepatocellular carcinoma, brain cancer (including glioma), pancreatic cancer, ovarian cancer, colorectal and/or colon cancer, breast cancer, prostate cancer, thyroid cancer, lung cancer, melanoma, or stomach cancer. In another embodiment, the cell proliferative disorder is glioma, pancreatic, ovarian, colorectal, breast, or prostate cancer. In one embodiment, the cell proliferative disorder is renal cancer. In one embodiment, the cell proliferative disorder is hepatocellular carcinoma. In yet another embodiment, the cell proliferative disorder is pancreatic cancer. In still another embodiment, the cell proliferative disorder is glioma. In one embodiment, the glioma is an anaplastic astrocytoma. In another embodiment, the glioma is a glioblastoma multiforme.

In one embodiment, the PAR-1 inhibitor is: 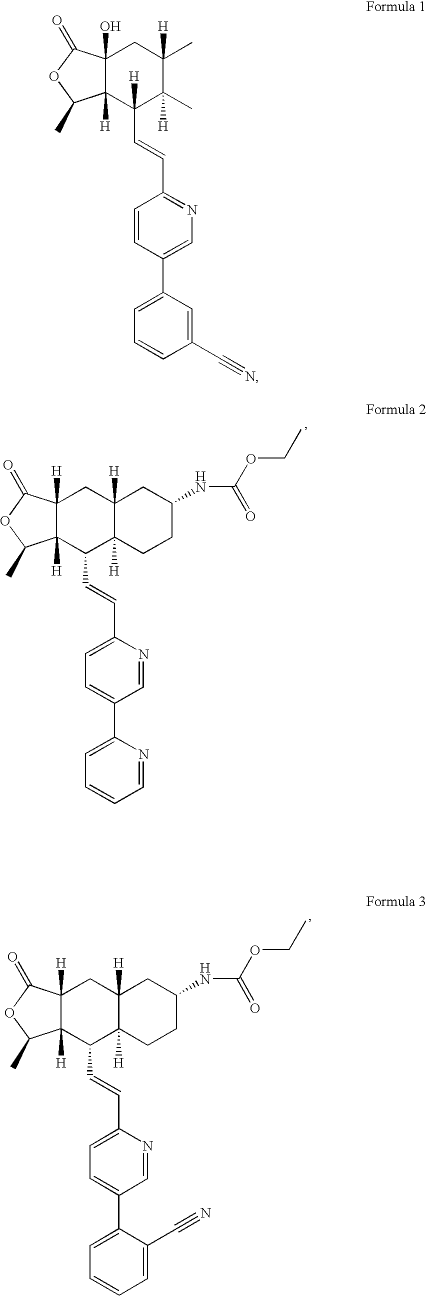 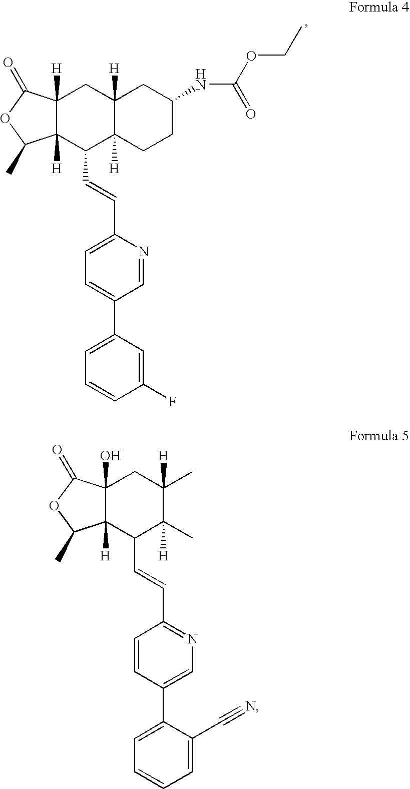
BMS-200261, RWJ-56110, RWJ-58259, a blocking antibody to PAR-1, a pepducin to PAR-1, an antisense oligonucleotide to the nucleic acid encoding PAR-1, a small interfering RNA or a short hairpin RNA to the mRNA encoding PAR-1, a pharmaceutically acceptable salt of any of the above, or a combination of two or more of the above.

In another embodiment, the PAR-1 inhibitor is: 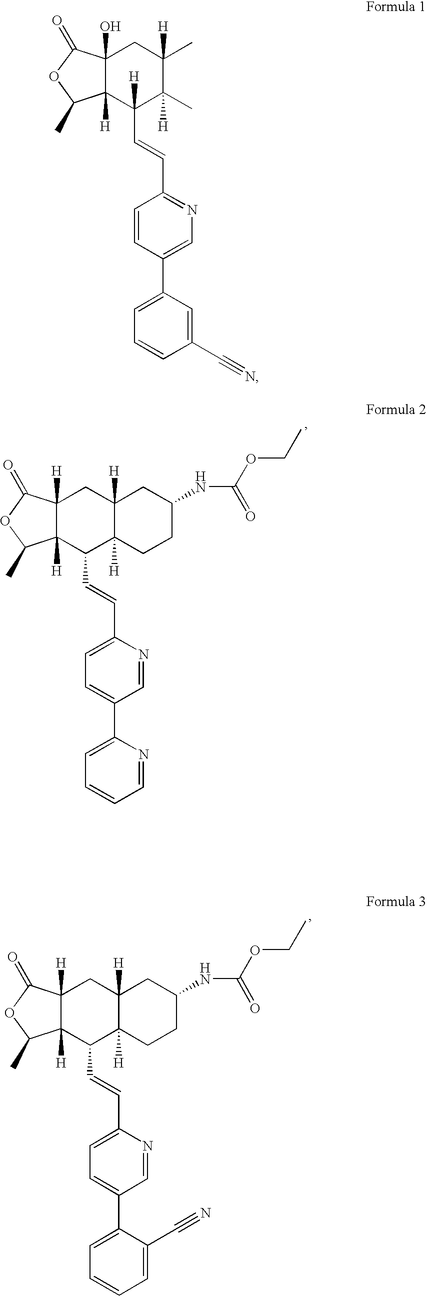 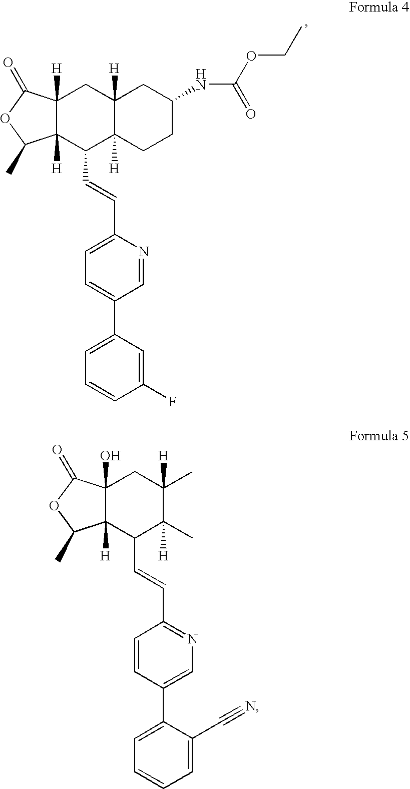
a pharmaceutically acceptable salt of any of the above, or a combination of two or more of the above.

In one embodiment, the PAR-1 inhibitor is Formula 1, or a pharmaceutically acceptable salt thereof. In another embodiment, the PAR-1 inhibitor is Formula 2, or a pharmaceutically acceptable salt thereof. In yet another embodiment, the PAR-1 inhibitor is Formula 3, or a pharmaceutically acceptable salt thereof.

In one embodiment of the methods detailed above, the PAR-1 inhibitor is administered in an amount sufficient to maintain the patient's plasma level of the PAR-1 inhibitor at or above 1 μM for 24 hrs.

In one embodiment, the methods detailed above further comprise administering another antineoplastic agent. In one embodiment, the other antineoplastic agent is temozolomide and the cell proliferative disorder is glioma. In another embodiment, the other antineoplastic agent is interferon and the cell proliferative disorder is melanoma. In one embodiment, the other antineoplastic agent is PEG-Intron (peginterferon alpha-2b) and the cell proliferative disorder is melanoma.

### DETAILED DESCRIPTION OF THE DRAWINGS

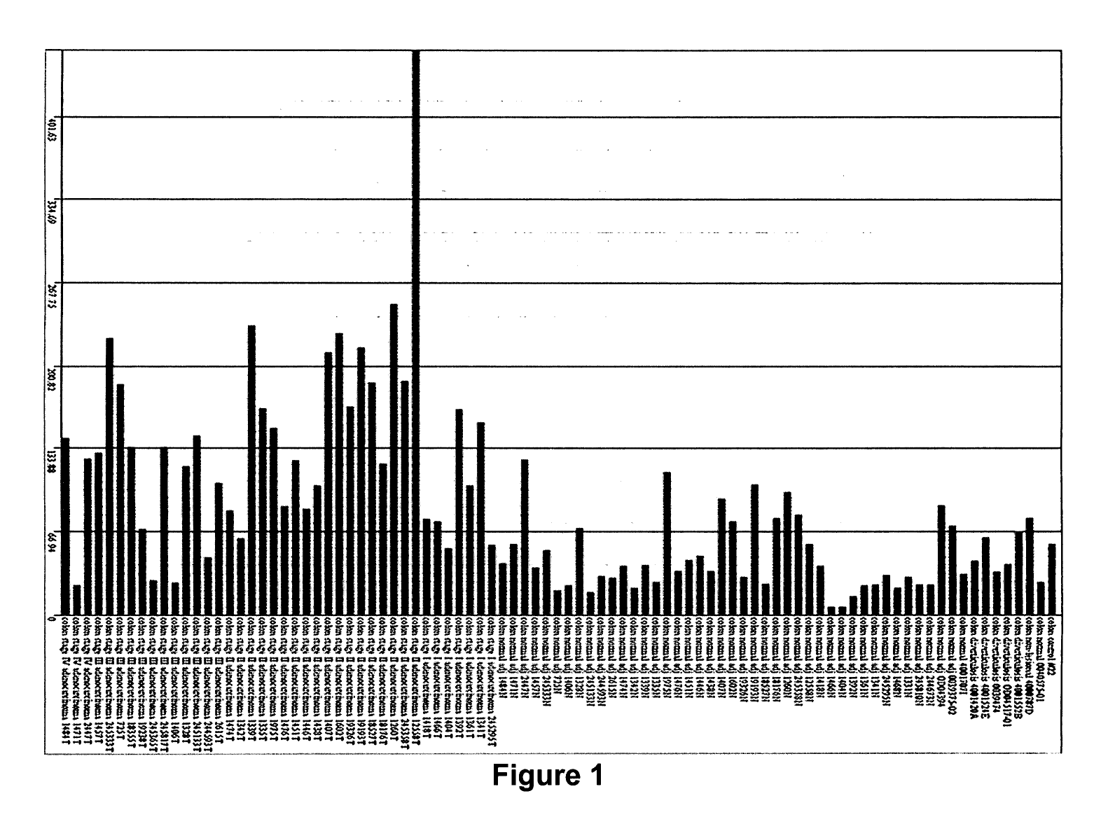

FIG. 1 illustrates the expression of PAR-1 mRNA among a sampling of human colorectal tumors and nonmalignant (“normal”) tissue.

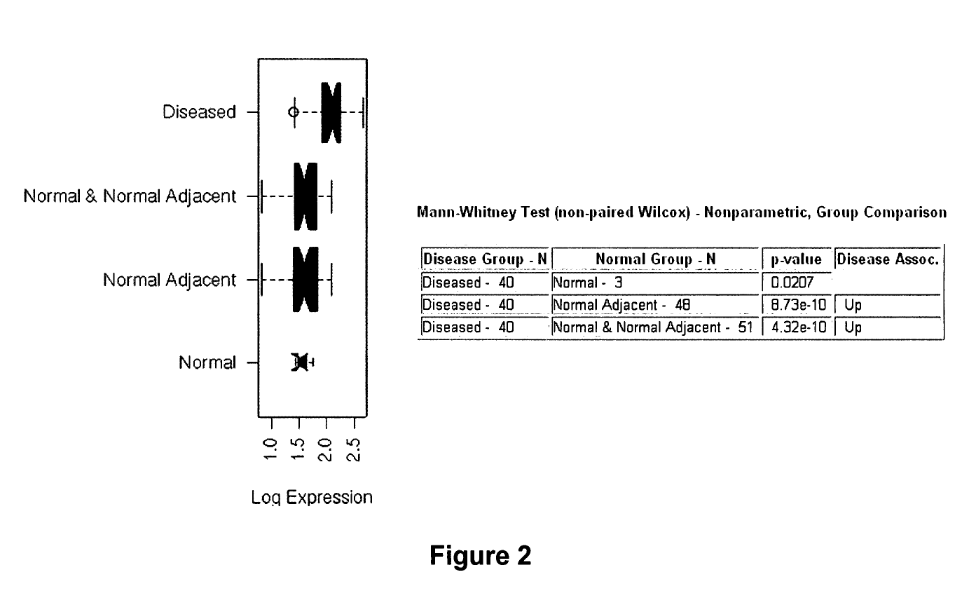

FIG. 2 illustrates the statistical differences in PAR-1 mRNA expression among a sampling of human colorectal tumor (“diseased”) samples as compared to nonmalignant (“normal” and “normal adjacent”) tissue.

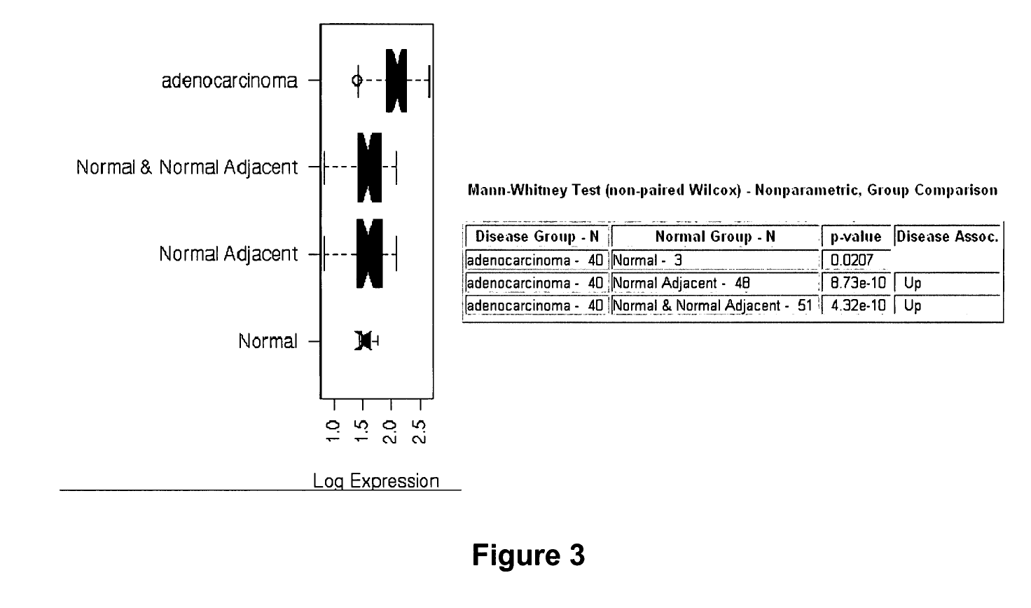

FIG. 3 illustrates the statistical differences in PAR-1 mRNA expression among a sampling of human colorectal tumor (“adenocarcinoma”) samples as compared to nonmalignant (“normal” and “normal adjacent”) tissue.

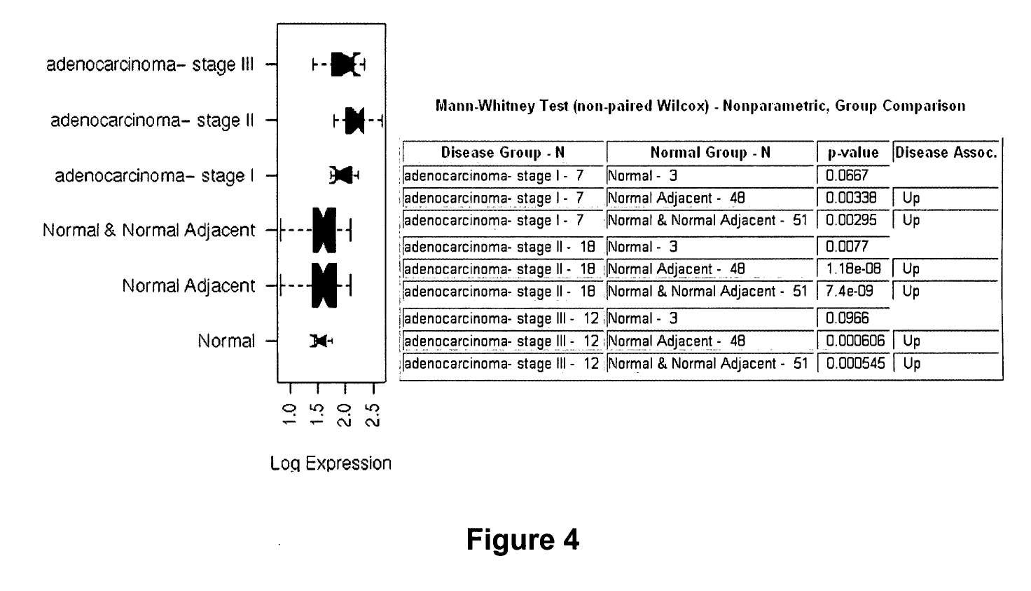

FIG. 4 illustrates the statistical differences in PAR-1 mRNA expression among a sampling of human colorectal tumor (“adenocarcinoma stage I, II, or III”) samples as compared to nonmalignant (“normal” and “normal adjacent”) tissue.

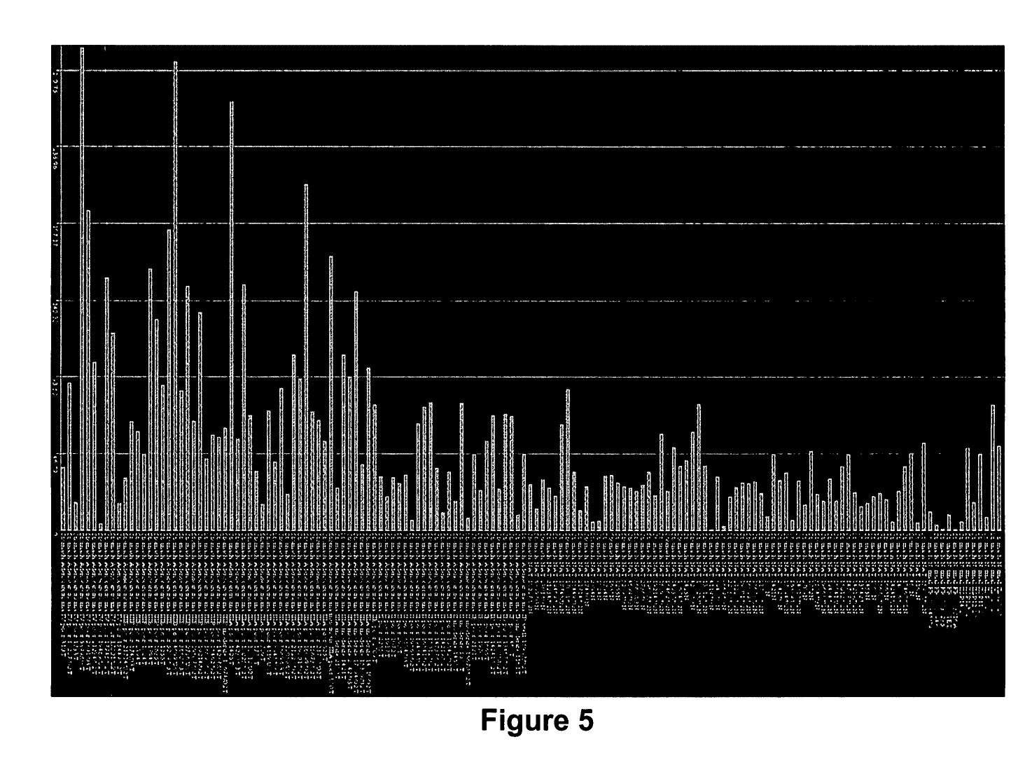

FIG. 5 illustrates the expression of PAR-1 mRNA among a sampling of human stomach tumors and nonmalignant (“normal”) tissue.

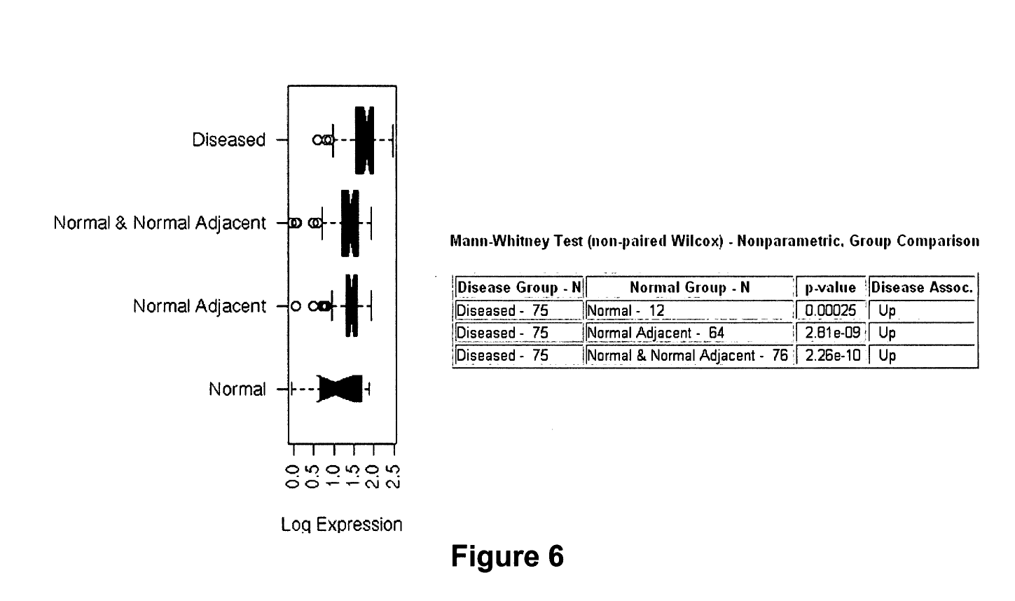

FIG. 6 illustrates the statistical differences in PAR-1 mRNA expression among a sampling of human stomach tumor (“diseased”) samples as compared to nonmalignant (“normal” and “normal adjacent”) tissue.

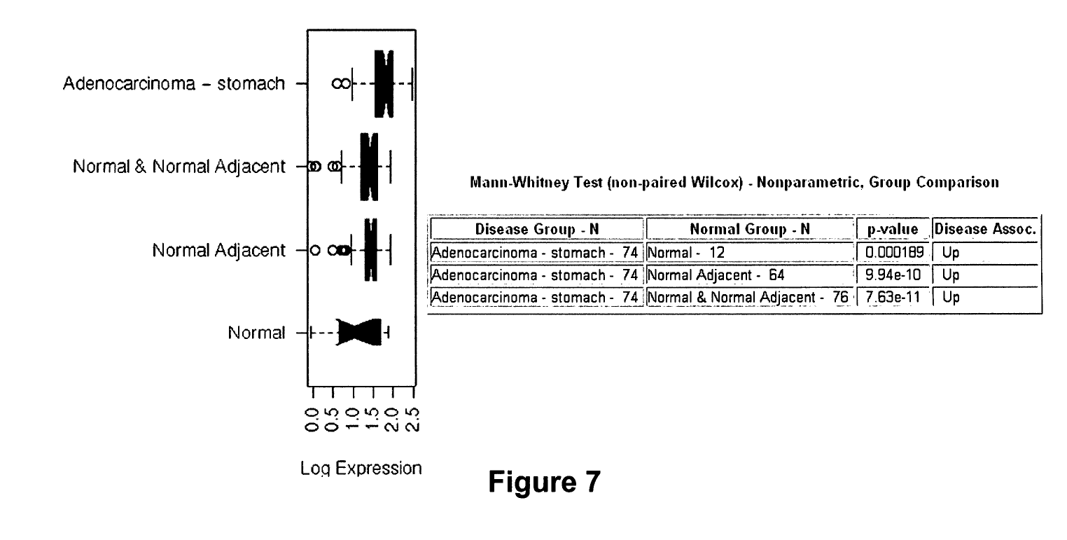

FIG. 7 illustrates the statistical differences in PAR-1 mRNA expression among a sampling of human stomach tumor (“adenocarcinoma”) samples as compared to nonmalignant (“normal” and “normal adjacent”) tissue.

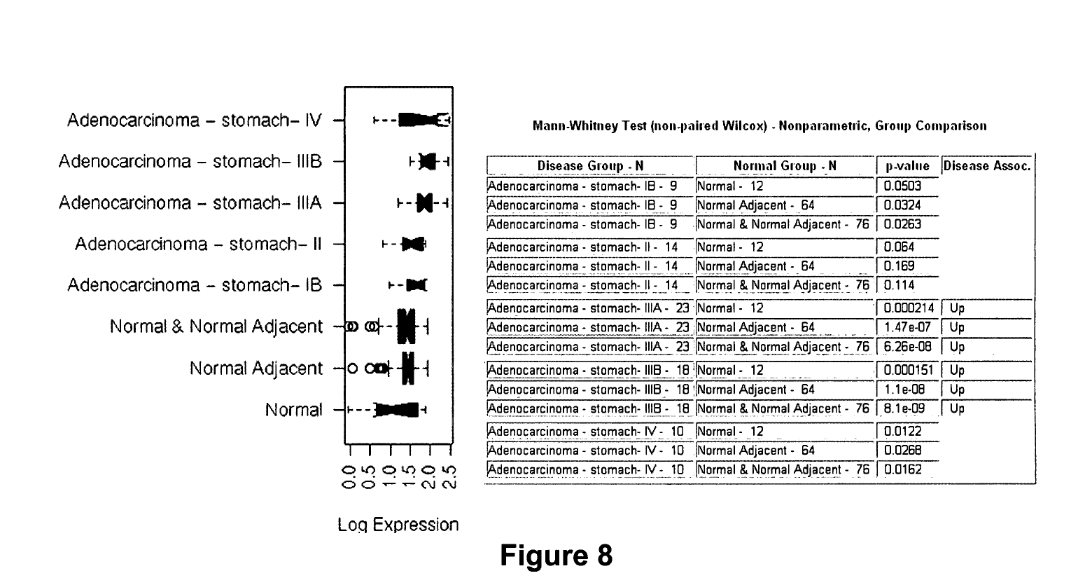

FIG. 8 illustrates the statistical differences in PAR-1 mRNA expression among a sampling of human stomach tumor (“adenocarcinoma stage IB, II, IIIA, IIIB, or IV”) samples as compared to nonmalignant (“normal” and “normal adjacent”) tissue.

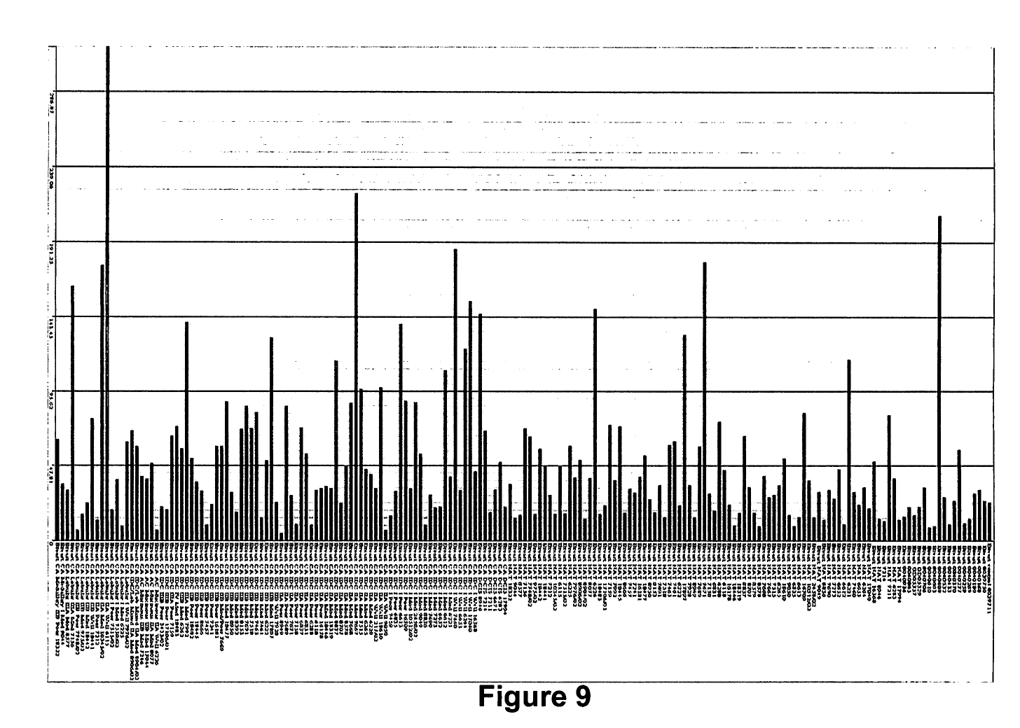

FIG. 9 illustrates the expression of PAR-1 mRNA among a sampling of human breast tumors and nonmalignant (“normal”) tissue.

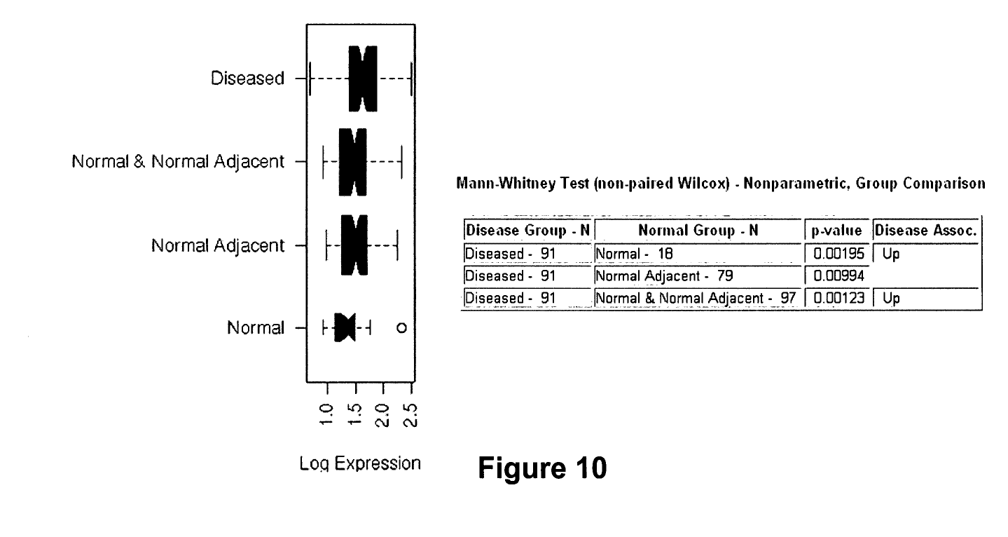

FIG. 10 illustrates the statistical differences in PAR-1 mRNA expression among a sampling of human breast tumor (“diseased”) samples as compared to nonmalignant (“normal” and “normal adjacent”) tissue.

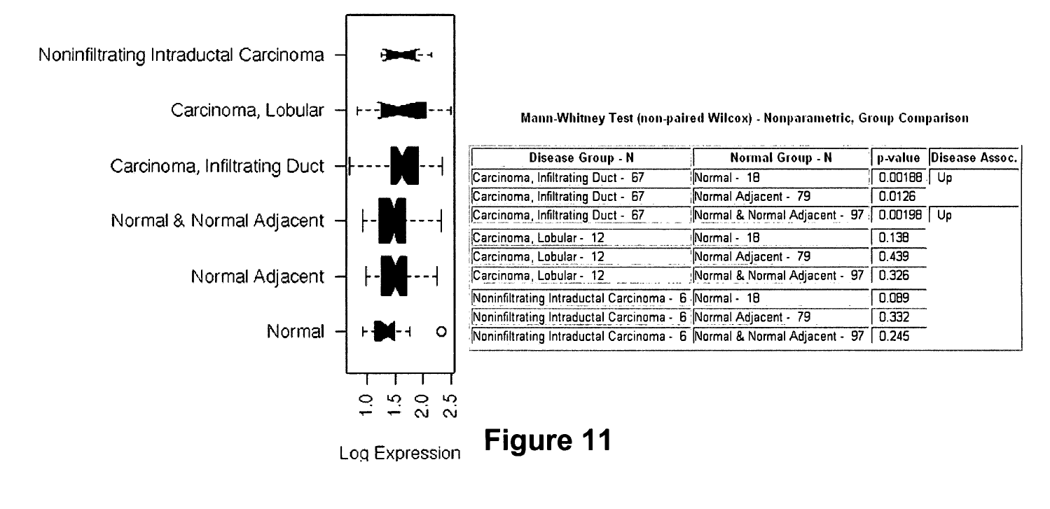

FIG. 11 illustrates the statistical differences in PAR-1 mRNA expression among a sampling of human breast tumor (“noninfiltrating intraductal carcinoma”; “carcinoma, lobular”; or “carcinoma, infiltrating duct”) samples as compared to nonmalignant (“normal” and “normal adjacent”) tissue.

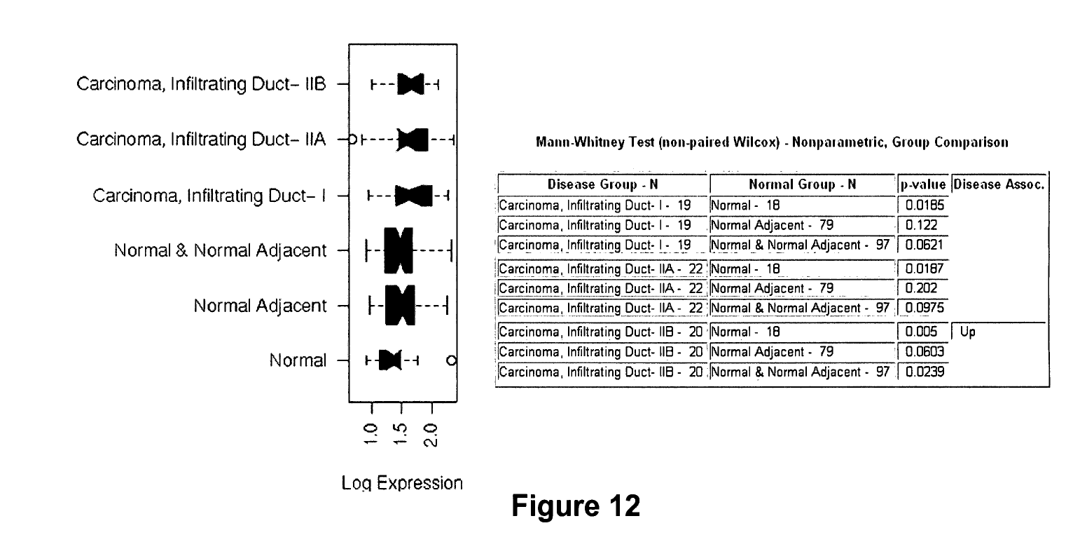

FIG. 12 illustrates the statistical differences in PAR-1 mRNA expression among a sampling of human breast tumor (“carcinoma, infiltrating duct stage I, IIA, or IIB”) samples as compared to nonmalignant (“normal” and “normal adjacent”) tissue.

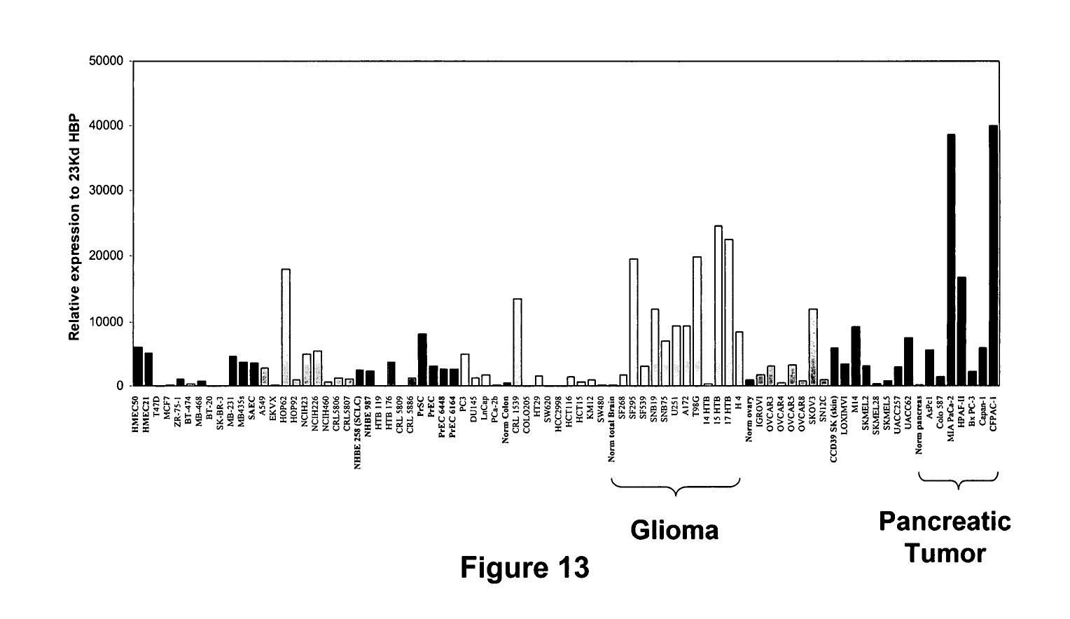

FIG. 13 illustrates the expression of PAR-1 mRNA among various human nonmalignant (“normal”) and tumor cell lines. Of note, the nonmalignant cell lines are in bold text.

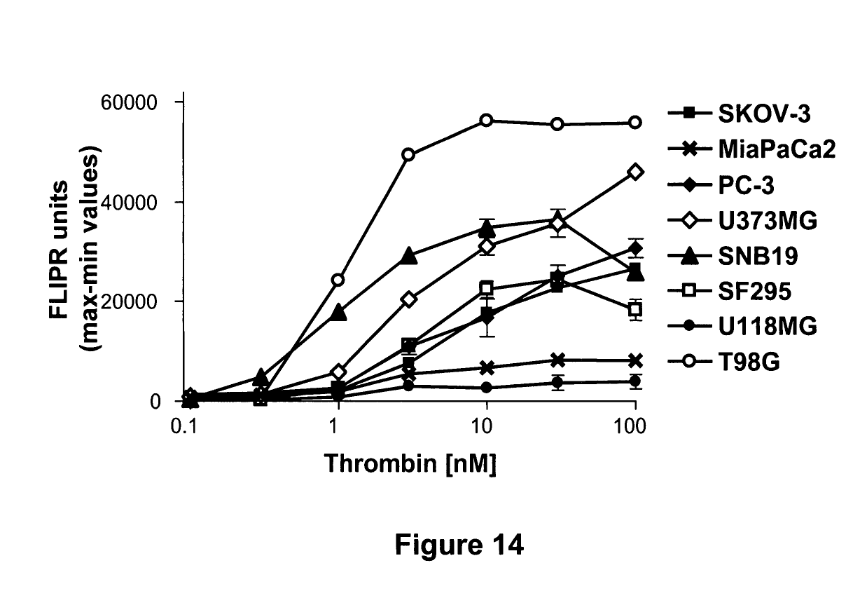

FIG. 14 illustrates intracellular Ca²⁺ release in cells from various tumor cell lines (i.e., the ovarian cancer cell lines SKOV-3; the pancreatic cancer cell line MiaPaCa2; the prostate cancer cell line PC-3; and glioma cell lines U373, SNB19, SF295, U118MG, and T98G) treated with thrombin (at concentrations of 0.1, 0.5, 1, 5, 10, 50, or 100 nM).

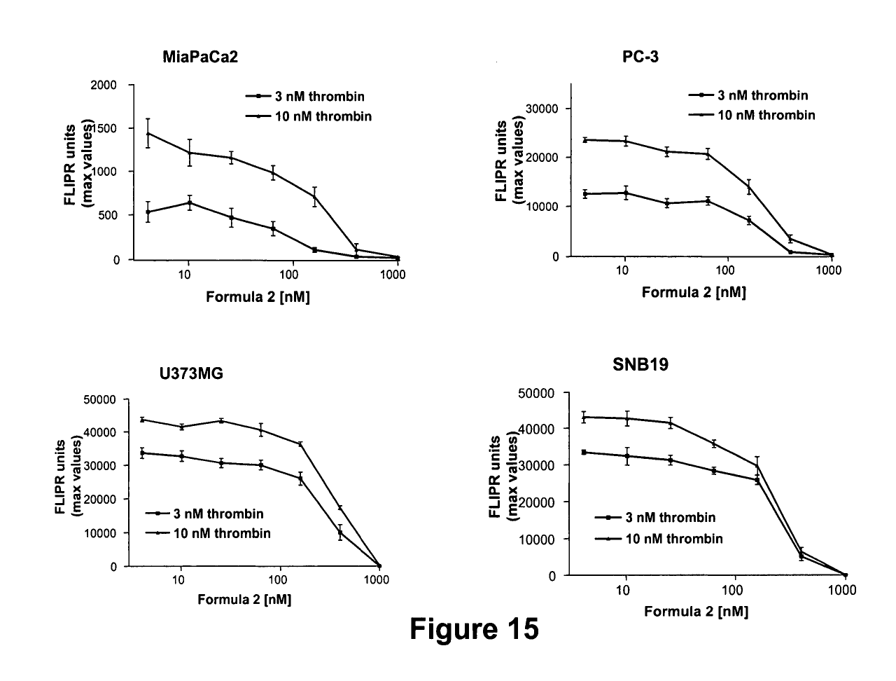

FIG. 15 illustrates intracellular Ca²⁺ release in cells from various cancer cell lines (i.e., the pancreatic cancer cell line MiaPaCa2; the prostate cancer cell line PC-3; and glioma cell lines U373 and SNB19), treated with thrombin (at concentrations of 3 nM or 10 nM) and PAR-1 inhibitor Formula 2 (at concentrations between 4-1000 nM).

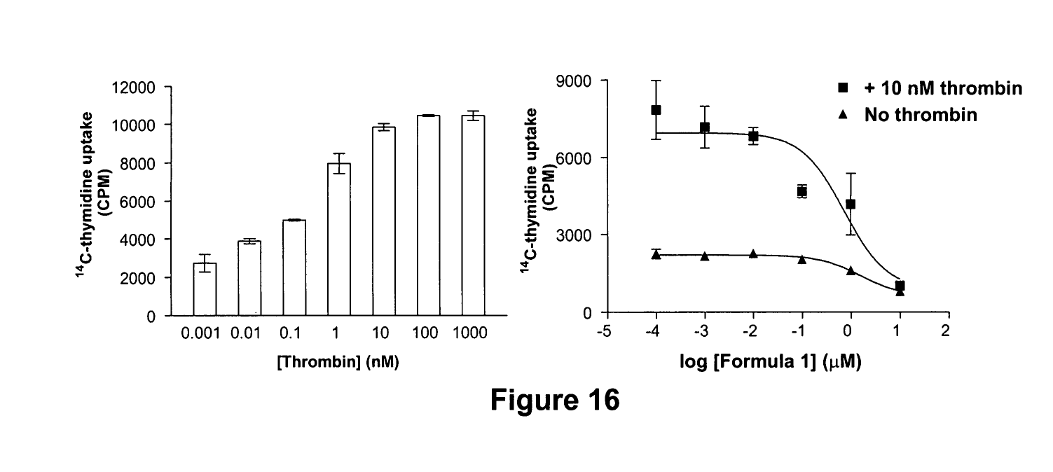

FIG. 16 illustrates ¹⁴C-thymidine incorporation in cells from the glioma cell line U373 treated with thrombin (at concentrations between 0.001-1000 nM) or with PAR-1 inhibitor Formula 1 (at concentrations between 10⁻⁴ to 10 μM) in the presence or absence of thrombin (at a concentration of 10 nM).

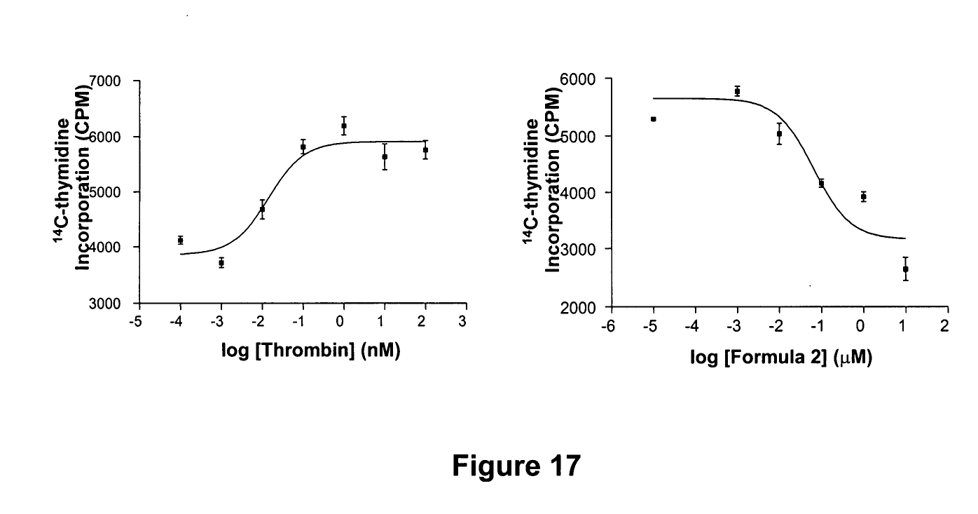

FIG. 17 illustrates ¹⁴C-thymidine incorporation in cells from the pancreatic cancer cell line MiaPaCa2 treated with thrombin (at concentrations between 10⁻⁴ to 10² nM) or with PAR-1 inhibitor Formula 2 (at concentrations between 10⁻⁵ to 10 μM) as well as thrombin (at a concentration of 1 nM).

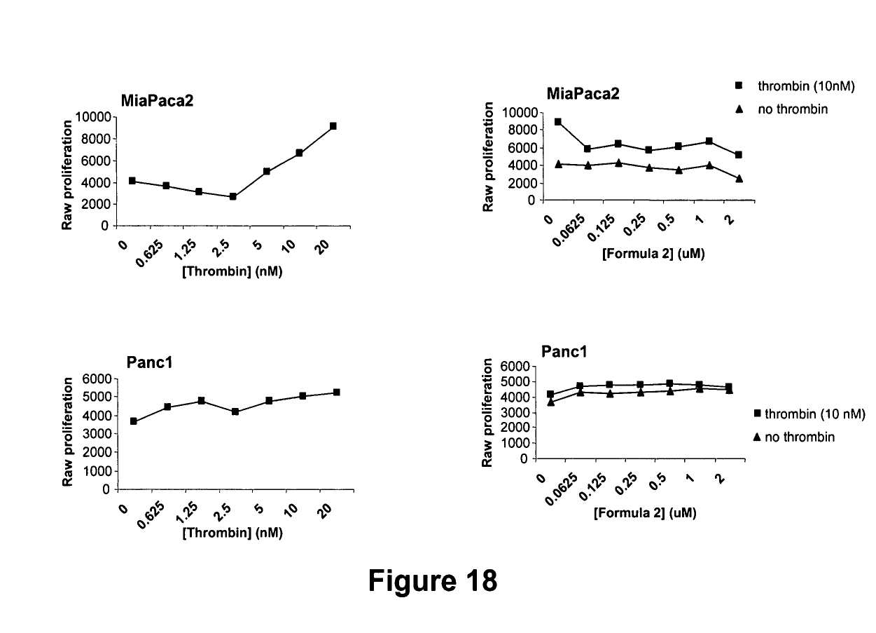

FIG. 18 illustrates the raw proliferation of cells (as measured using the calcein AM assay) from the pancreatic cancer cell lines MiaPaCa2 and Panc1 treated with thrombin, or PAR-1 inhibitor Formula 1 in the presence or absence of thrombin (at a concentration of 10 nM).

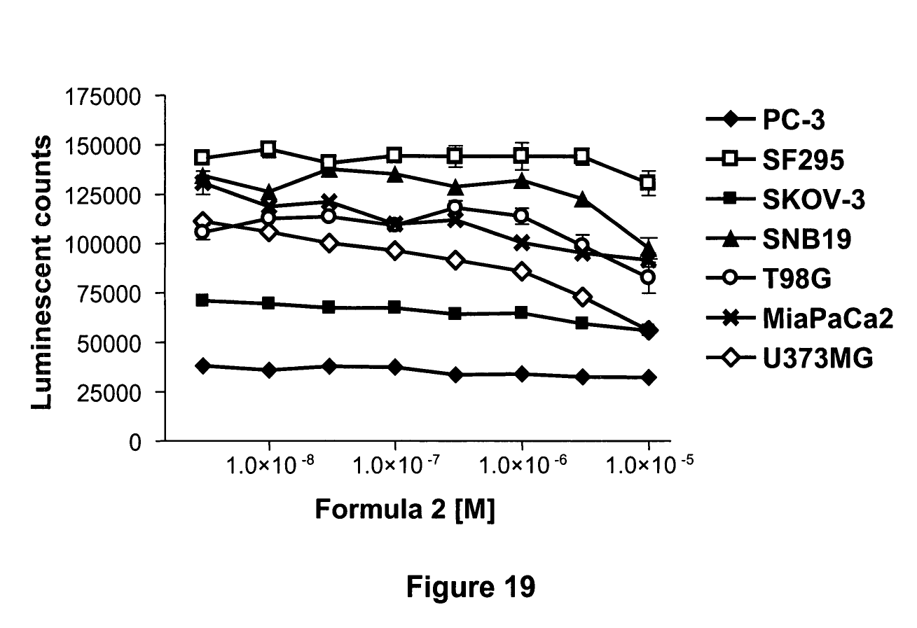

FIG. 19 illustrates cell proliferation (as measured by luminescent counts in a Cell-TiterGlo assay) in various cell lines (i.e., the ovarian cancer cell lines SKOV-3; the pancreatic cancer cell line MiaPaCa2; the prostate cancer cell line PC-3; and glioma cell lines U373, SNB19, SF295, and T98G) treated with PAR-1 inhibitor Formula 2.

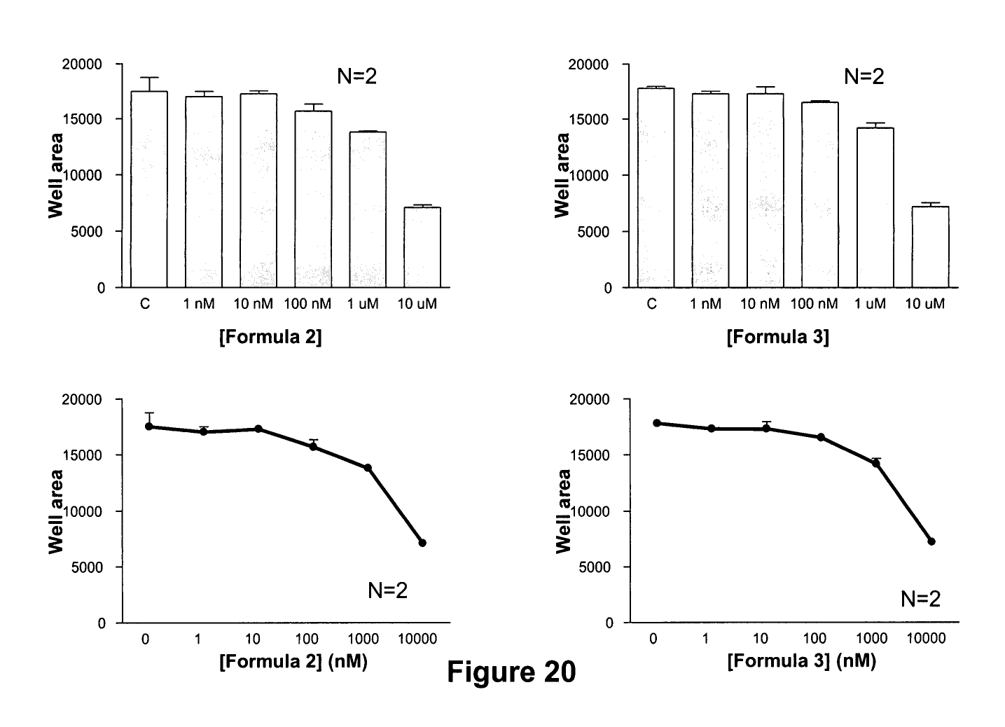

FIG. 20 illustrates cell proliferation (as measured by soft agar assay) in the pancreatic cancer cell line MiaPaCa2 treated with PAR-1 inhibitor Formula 2 (at concentrations between 0-10,000 nM) or PAR-1 inhibitor Formula 3 (at concentrations between 0-10,000 nM).

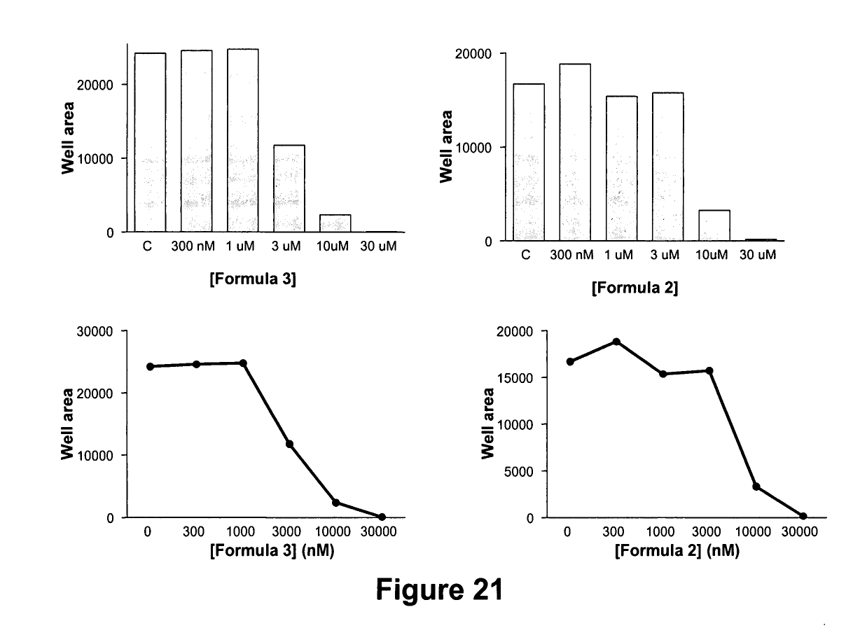

FIG. 21 illustrates cell proliferation (as measured by soft agar assay) in the glioma cancer cell line SF295 treated with PAR-1 inhibitor Formula 2 (at concentrations between 0-30,000 nM) or PAR-1 inhibitor Formula 3 (at concentrations between 0-30,000 nM).

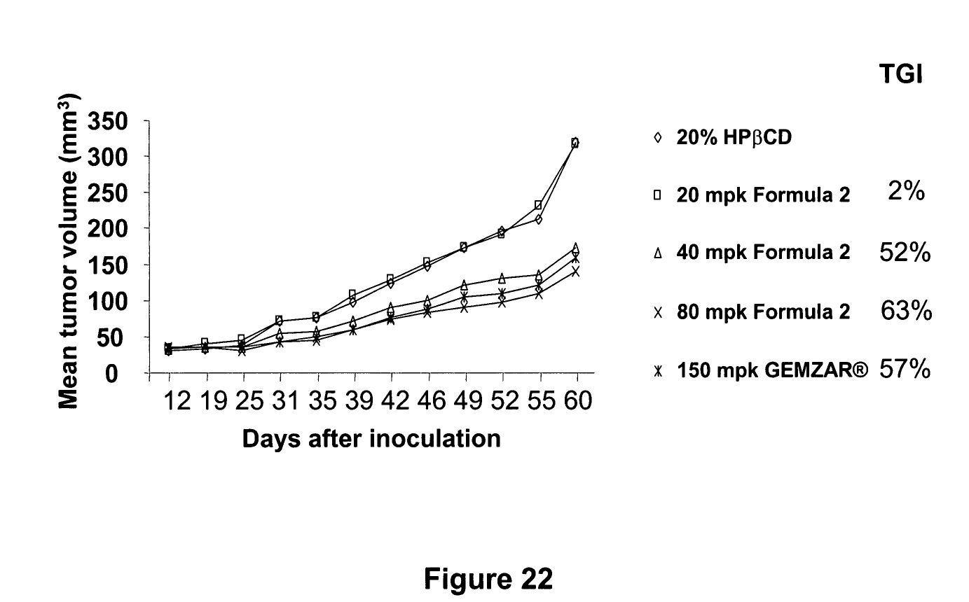

FIG. 22 illustrates the mean tumor growth curves of xenograft tumors formed using the pancreatic cancer cell line MiaPaCa2 following treatment with control vehicle (i.e., 20% hydroxypropyl-beta-cyclodextrin (HP-β-CD)); PAR-1 inhibitor Formula 2 at doses of 20 mg per kg (mpk), 40 mpk, or 80 mpk; or GEMZAR® (gemcitabine HCl) at a dose of 150 mpk. In addition, the percentage of tumor growth inhibition (TGI) in xenografts following treatment with PAR-1 inhibitor Formula 2 or GEMZAR® (gemcitabine HCl) is noted.

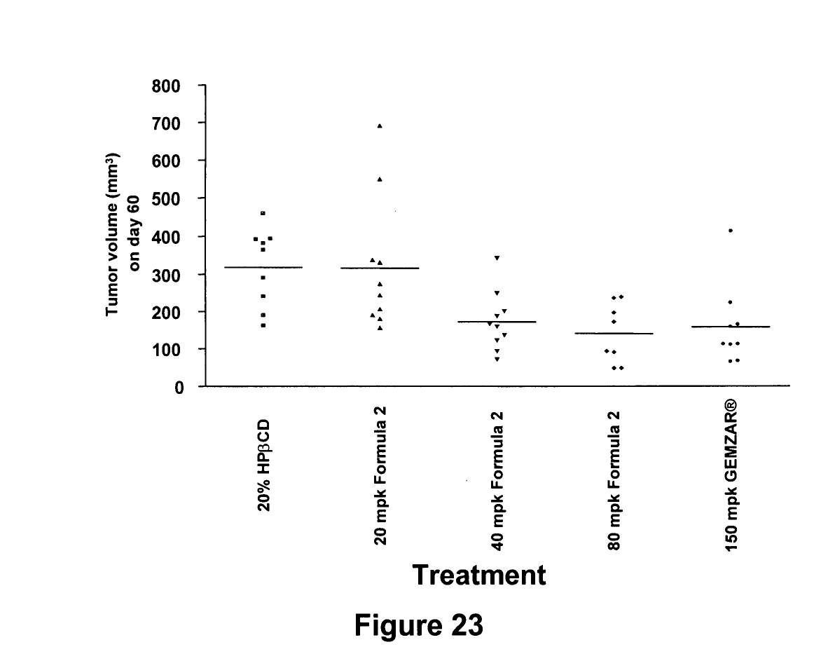

FIG. 23 illustrates the tumor volume on Day 60 of individual xenograft tumors formed using the pancreatic cancer cell line MiaPaCa2 following treatment with control vehicle (i.e., 20% HP-β-CD); PAR-1 inhibitor Formula 2 at doses of 20 mpk, 40 mpk, or 80 mpk; or GEMZAR® (gemcitabine HCl) at a dose of 150 mpk.

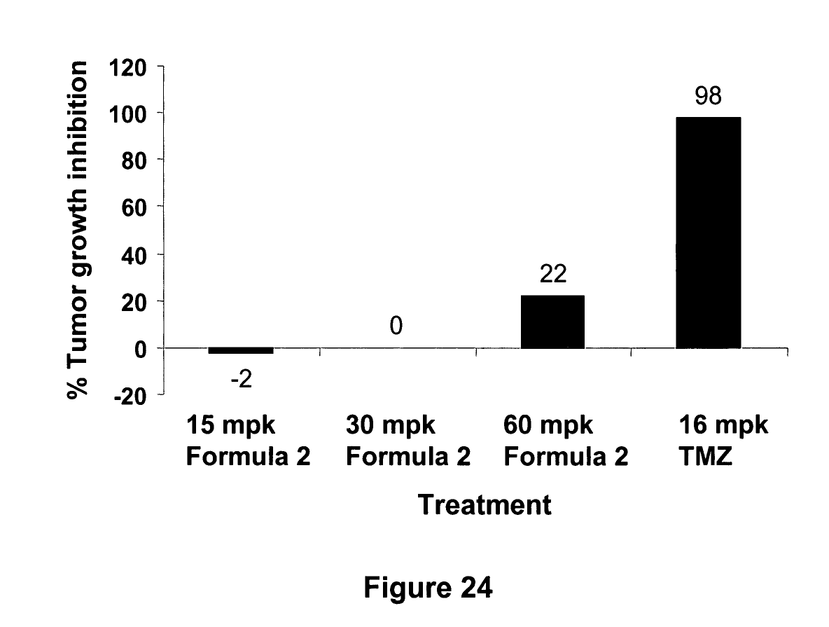

FIG. 24 illustrates the percentage of tumor growth inhibition in xenograft tumors formed using the glioma cell line U373 following treatment with PAR-1 inhibitor Formula 2 at doses of 15 mpk, 30 mpk, or 60 mpk; or temozolomide (TMZ) at a dose of 16 mpk.

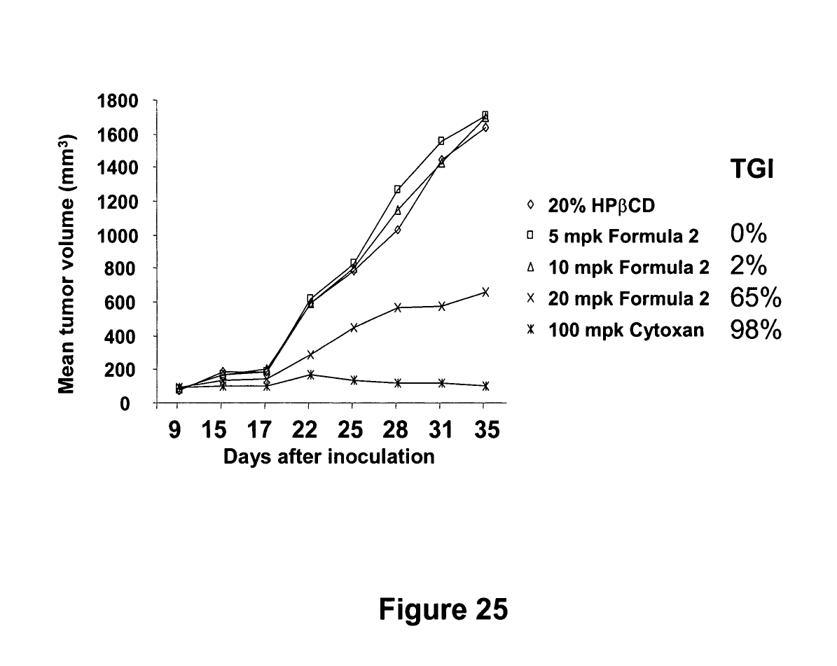

FIG. 25 illustrates the mean tumor growth curves of xenograft tumors formed using the breast cancer cell line MDA-MB-231 following treatment with control vehicle (i.e., 20% HP-β-CD); PAR-1 inhibitor Formula 2 at doses of 5 mpk, 10 mpk, or 20 mpk; or Cytoxan at a dose of 100 mpk. In addition, the percentage of tumor growth inhibition (TGI) following treatment with PAR-1 inhibitor Formula 2 or Cytoxan is noted.

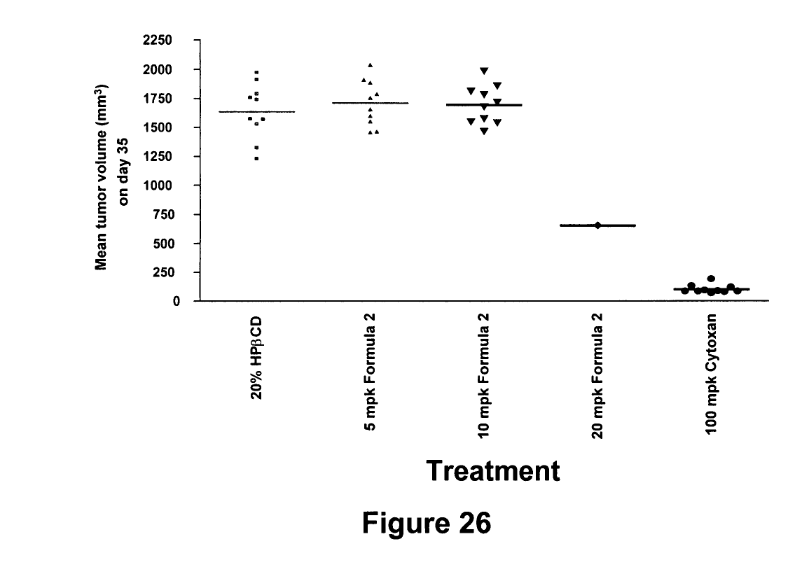

FIG. 26 illustrates the tumor volume on Day 35 of individual xenograft tumors formed using the breast cancer cell line MDA-MB-231 following treatment with control vehicle (i.e., 20% HP-β-CD)); PAR-1 inhibitor Formula 2 at doses of 5 mpk, 10 mpk, or 20 mpk; or Cytoxan at a dose of 100 mpk.

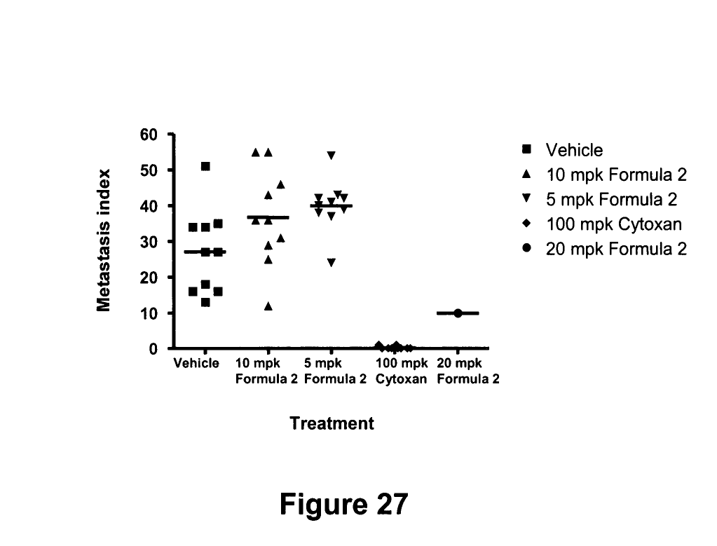

FIG. 27 illustrates lung metastasis in mice from xenograft tumors formed using the breast cancer cell line MDA-MB-231 following treatment with control vehicle (i.e., 20% HP-β-CD)); PAR-1 inhibitor Formula 2 at doses of 5 mpk, 10 mpk, or 20 mpk; or Cytoxan at a dose of 100 mpk. Of note, 9 of the 10 mice treated with PAR-1 inhibitor Formula 2 at a dose of 20 mpk did not survive.

### DETAILED DESCRIPTION OF THE INVENTION

As used herein, the following terms shall have the definitions set forth below.

As used herein, the phrase “protease activated receptor-1 inhibitor” also referred to herein as “PAR-1,” means an agent that inhibits signaling from protease activated receptor-1. An exemplary assay for identifying PAR-1 inhibitors (filtration binding assay) is described in Ahn et al., Mol Pharmacol, 51:350-356 (1997). Briefly, human platelet membranes (40 μg/0.2 ml reaction mixture) are incubated with 10 nM [³H]haTRAP and various concentrations of test compound at room temperature for 1 hour. Bound and free radioactivity are separated by rapid vacuum-assisted filtration and the bound radioactivity is quantified by liquid scintillation counting. Curve fitting is performed and the concentration of a test compound to displace 50% of specific binding is determined.

As used herein, the phrase “therapeutically effective amount” with respect to a PAR-1 inhibitor means an amount which provides a therapeutic benefit in the prevention and/or treatment or management of the referenced cell proliferative disorder (e.g., pancreatic cancer).

As used herein the phrase “pharmaceutically acceptable salt” refers to a non-toxic salt prepared from a pharmaceutically acceptable acid or base (including inorganic acids or bases, or organic acids or bases). Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine, and procaine.

As used herein, the phrase “cell proliferative disorder” refers to a neoplasm. That is, a new, abnormal growth of cells or a growth of abnormal cells which reproduce faster than normal. A neoplasm creates an unstructured mass (a tumor) which can be either benign or malignant. The term “benign” refers to a tumor that is noncancerous, e.g., its cells do not invade surrounding tissues or metastasize to distant sites. The term “malignant” refers to a tumor that is cancerous, and/or metastastic, i.e., invades contiguous tissue or is no longer under normal cellular growth control. A neoplasm can be classified by its histological appearance or by the presumptive organ of the primary or putative cell of origin. A neoplasm can be classified by its histological appearance based on the type of cell that it resembles and, therefore, the tissue presumed to be the origin of the tumor. The following are general categories used to classify malignant neoplasms:
Carcinoma: derived from epithelial cells
Lymphoma and Leukemia: derived from blood and bone marrow cells
Sarcoma: derived from connective tissue, or mesenchymal cells
Mesothelioma: derived from the mesothelial cells lining the peritoneum and the pleura
Glioma: derived from glia, the most common type of brain cell
Germinoma: derived from germ cells, normally found in the testicle and ovary
Choriocarcinoma: derived from the placenta

In preferred embodiments, the methods of the invention are used to prevent and/or treat cell proliferative disorders including but not limited to head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, marginal zone lymphoma, Burkitt Lymphoma, mycosis fungoides, seary syndrome, cutaneous T-cell lymphoma, peripheral T cell lymphoma, chronic myeloproliferative disorders, myelofibrosis, myeloid metaplasia, systemic mastocytosis), and central nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelofibrosis), soft tissue sarcoma, thyroid cancer, endometrial cancer, carcinoid cancer, or liver cancer. In one preferred embodiment, the methods of the invention are used to prevent and/or treat cell proliferative disorders including but not limited to carcinoma (including breast cancer, prostate cancer, lung cancer, colorectal and/or colon cancer, hepatocellular carcinoma, melanoma), lymphoma (including non-Hodgkin's lymphoma and mycosis fungoides), leukemia, sarcoma, mesothelioma, brain cancer (including glioma), germinoma (including testicular cancer and ovarian cancer), choriocarcinoma, renal cancer, pancreatic cancer, thyroid cancer, head and neck cancer, endometrial cancer, cervical cancer, bladder cancer, or stomach cancer. In more preferred embodiments, the methods of the invention are used to prevent and/or treat renal cancer, hepatocellular carcinoma, brain cancer (including glioma), or pancreatic cancer. In a most preferred embodiment, the cell proliferative disorder is pancreatic cancer.

Preferably, assuming a patient having a bodyweight of 70 kg, an exemplary dosing regimen for a PAR-1 inhibitor is QD: up to 4000 mg. For example, a preferred dosing regimen for a PAR-1 inhibitor (e.g., Formula 2) is as follows, QD: 900 mg to 4000 mg, more preferably 2400 mg; BID: 284 mg to 392 mg, more preferably 338 mg; or TID: 224 mg to 352 mg, more preferably 288 mg. Preferably, the dosing regimen maintains the patient's plasma level of PAR-1 inhibitor at or above 1 μM for 24 hrs.

The dosing regimen for a PAR-1 inhibitor may be administered by various routes including but not limited to, oral (p.o.), intraperitoneal (i.p.), intravascular (i.v.), subcutaneous (s.c.), or intrathecal (i.t.) routes of administration.

The amount and frequency of administration of the compounds of the invention and/or the pharmaceutically acceptable salts thereof will be regulated according to the judgment of the attending clinician considering such factors as age, condition and size of the patient as well as severity of the symptoms being treated or the patient's disposition to cancer.

In another embodiment, the PAR-1 inhibitor is selected from the group consisting of Formula 1, Formula 2, Formula 3, Formula 4, Formula 5, BMS-200261 (Bernatowicz et al., 39(25):4879-4887 (1996)), RWJ-56110 (Maryanoff et al., Curr Med Chem Cardiovasc Hematol Agents, 1(1): 13-36 (2003)), and RWJ-58259 (Maryanoff et al., Curr Med Chem Cardiovasc Hematol Agents, 1(1):13-36 (2003)), a blocking antibody to PAR-1 (Kahn et al., Clin Invest, 103(6):879-887 (1999)), a pepducin (cell-penetrating peptide) to PAR-1 (Covic et al., Proc Natl Acad Sci USA, 99(2):643-648 (2002), an antisense oligonucleotide to the nucleic acid encoding PAR-1, a small interfering RNA or a short hairpin RNA to the mRNA encoding PAR-1, a pharmaceutically acceptable salt of any of the above, and a combination of two or more of the above.

Also encompassed within the scope of the present invention are methods of administering one or more PAR-1 inhibitors in combination with another antineoplastic agent. Non-limiting examples of other useful antineoplastic agents include Uracil Mustard, Chlormethine, Cyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Methotrexate, 5-Fluorouracil, Floxuridine, Cytarabine, 6-Mercaptopurine, 6-Thioguanine, Fludarabine phosphate, Pentostatine, Vinblastine, Vincristine, Vindesine, Bleomycin, Dactinomycin, Daunorubicin, Doxorubicin, Epirubicin, Idarubicin, Paclitaxel, Mithramycin, Deoxycoformycin, Mitomycin-C, L-Asparaginase, Interferons, Etoposide, Teniposide 17.alpha.-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Oxaliplatin (Eloxatin®), Iressa (gefinitib, Zd1839), XELODA® (capecitabine), Tarceva® (erlotinib), Azacitidine (5-Azacytidine; 5-AzaC), temozolomide (Temodar®), Gemcitabine (e.g., GEMZAR® (gemcitabine HCl)), vasostatin, or a combination of two or more of the above.

In one embodiment, the present invention provides methods of administering one or more PAR-1 inhibitors in combination with temozolomide (Temodar®; see, e.g., U.S. Pat. No. 5,260,291) in the prevention and/or treatment of glioma.

In another embodiment, the present invention provides methods of administering one or more PAR-1 inhibitors in combination with interferon (e.g., PEG-Intron (peginterferon alpha-2b); see, e.g., EP 1 043 026) for the prevention and/or treatment of melanoma.

### EXAMPLES

Assay for mRNA Expression of PAR-1

As detailed below, mRNA expression of PAR-1 was assayed in human tumor cells as well as human tumor cell lines. Of note, all data points were normalized to an endogenous control, human 23 kD highly basic protein (HBP), whose mRNA expression remained relative consistent across the test samples assayed. In brief, total RNA was isolated using a CsCl purification method and treated with DNase I (Boehringer-Mannheim, Indianapolis, Ind.) to remove residual DNA. The relative mRNA level of human PAR-1 gene or HBP gene, was measured with the TaqMan 5′ nuclease quantitative RT-PCR assay. Taqman EZ RT-PCR kit (PE Applied Biosystems, Foster City, Calif.) was used to perform the quantitative RT-PCR on 7700 Sequence Detector (PE Applied Biosystems, Foster City, Calif.). The primers for PAR-1 ((forward primer 5′-CCAACCGCAGCAAGAAGTC-3′ (SEQ ID NO: 1); reverse primer 5′-GCAAATGATGAAGATGCAGAAAA-3′ (SEQ ID NO: 2)) and probe for PAR-1 (5′-CGGGCTTTGTTCCTGTCAGCTGCT-3′ (SEQ ID NO: 3)) were obtained from PE Applied Biosystems (Foster City, Calif.). The primers for 23 kD HBP (forward primer 5′-CTGGAAGTACCAGGCAGTGACA-3′ (SEQ ID NO: 4); reverse primer 5′-CCACCCTGGAGGAGAAGAGGAAAGAGAA-3′ (SEQ ID NO: 5)) and probe for 23 kD HBP (5′-CCGGTAGTGGATCTTGGCTTT-3′ (SEQ ID NO: 6)) were obtained from PE Applied Biosystems (Foster City, Calif. Each probe was labeled with a reporter fluorescent dye [FAM (6-carboxyl-fluorescein)] at the 5′ end, and a quencher fluorescent dye TAMRA (6-carboxyl-tetramethyl-rhodamine) at the 3′ end.

Human Tumors

The mRNA expression of PAR-1 was measured in various human tumors. Tumor tissues were collected for each case, and when possible matching normal adjacent tissue was also collected. All tissues were screened in-house by pathologists to confirm staging diagnoses. Total RNA was prepared from tissue by standard methodologies and reverse transcribed. Real-time quantitative PCR was performed by standard methodologies. The absence of genomic DNA contamination was confirmed using primers that recognize a genomic region of the CD4 promoter. Ubiquitin levels were measured in a separate reaction and used to normalize the data by the Δ-Δ Ct method. See User Bulletin #2 (1997) Applied Biosystems, Foster City, Calif. (Using the mean cycle threshold value for ubiquitin and MDL-1 for each sample, the equation 1.8 e (Ct ubiquitin minus Ct PAR-1)×10⁴ was used to obtain the normalized values.) Mann-Whitney non-parametric non-paired Wilcox statistical analysis was performed on log transformed data (median method). See Hollander and Wolfe (1973) Nonparametric Statistical Interference, John Wiley and Sons, New York, N.Y., pp. 115-120.

As illustrated in FIGS. 1-12, mRNA expression of PAR-1 was elevated in human colorectal tumors, human stomach tumors, and human breast tumors as compared to non-malignant (i.e., “normal” and “normal adjacent”) tissues.

Human Colorectal Tumor Panel

The colorectal tumor panel included 11 control colon tissues; 40 normal adjacent tissues, matched with tumor cases; and 40 colorectal adenocarcinoma tissues, ordered by stage/differentiation on panel.

The control tissues (n=11) had a median PAR-1 expression value of 39.23, while the colorectal tumor samples of stage I, stage II, and stage III/IV had median PAR-1 expression values of 77.01 (1.96 fold), 166.43 (4.24 fold), and 124.94 (3.18 fold), respectively. Mann-Whitney non-parametric, non-paired Wilcox median analysis on log-transformed data showed statistically significant elevation of PAR-1 expression in all three stage groups of colorectal adenocarcinomas, with P values of less than 0.005 for all three groups, compared to normal control plus normal adjacent tissues (n=51).

Human Stomach Tumor Panel

The stomach tumor panel included 12 control stomach tissues; 64 normal adjacent tissues, matched with tumor cases (where available); and 75 stomach adenocarcinoma tissues, ordered by stage/differentiation on panel.

The control tissues (n=12) had a median PAR-1 expression value of 26.84, while the stomach tumor samples of stage I, stage II, stage IIIA, stage IIIB and stage IV had median PAR-1 expression values of 46.91 (1.7 fold), 33.75 (1.25 fold), 72.93 (2.8 fold), 66.95 (2.49 fold), and 96.17 (3.58 fold), respectively. Mann-Whitney non-parametric, non-paired Wilcox median analysis on log-transformed data showed statistically significant elevation of PAR-1 expression in stage IIIA and IIIB stomach adenocarcinomas, compared to normal control plus normal adjacent tissues (n=76) with P values of less than 0.0002.

Human Breast Tumor Panel

The breast tumor panel included 18 control breast tissues; 79 normal adjacent tissues, matched with tumor cases (where available); and 91 breast tumor cases: 6 ductal carcinoma in situ, 64 infiltrating ductal carcinoma (IDC), 4 mucinous IDC, 2 mixed IDC, 13 infiltrating lobular carcinoma and 2 medullary carcinoma tissues, ordered by stage/differentiation on the panel.

The control tissues (n=18) had a median PAR-1 expression value of 30.04, while the breast IDC tumor samples of stage I, stage II and stage III/IV and lobular (all stages) had median PAR-1 expression values of 42.16 (1.4 fold), 43.63 (1.45 fold), 62.88 (2.1 fold), and 38.63 (1.28 fold), respectively. Data from groups with small “n” were excluded from statistical analysis (mucinous IDC, mixed IDC and medullary carcinomas). Mann-Whitney non-parametric, non-paired Wilcox median analysis on log-transformed data showed statistically significant elevation of PAR-1 expression in all grouped IDC samples compared to the control tissues (n=97), with a P value of 0.0019.

Human Tumor Cell Lines

Similarly, as illustrated in FIG. 13, mRNA expression of PAR-1 was found to be elevated in various human tumor cell lines, including pancreatic tumor and glioma cell lines, relative to non-malignant (i.e., “normal”) human cell lines.

Intracellular [Ca²⁺] Release (FLIPR) Assay

As detailed below, intracellular Ca²⁺ release was measured in various tumor cell lines treated with thrombin. Likewise, intracellular Ca²⁺ release was measured in a subset of those tumor cell lines treated with thrombin and PAR-1 inhibitor Formula 2. In brief, cells were plated into a 96-well plate at a density of 4×10⁴ cells per well and allowed to attach overnight at 37° C. prior to loading with Fluo-4AM. Cells were then incubated at 37° C. for 60 minutes. After washing the cells, thrombin as well as the appropriate concentration of PAR-1 inhibitor Formula 2 was added and cells incubated at 37° C. for 30 minutes prior to taking fluorescence readings.

As illustrated in FIG. 14, thrombin stimulates intracellular Ca²⁺ release in cells from various tumor cell lines (i.e., the ovarian cancer cell lines SKOV-3; the pancreatic cancer cell line MiaPaCa2; the prostate cancer cell line PC-3; and glioma cell lines U373, SNB19, SF295, U118MG, and T98G).

As illustrated in FIG. 15, PAR-1 inhibitor Formula 2 inhibits intracellular Ca²⁺ release in cells from various tumor cell lines (i.e., the pancreatic cancer cell line MiaPaCa2; the prostate cancer cell line PC-3; and glioma cell lines U373 and SNB19).

¹⁴C-thymidine Incorporation Assay

Cell growth proliferation was ascertained by measuring ¹⁴C-thymidine incorporation in various cell lines treated with thrombin in the presence or absence of a PAR-1 inhibitor. In brief, 5×10³ cells per well were seeded in a 96-well plate and incubated at 37° C. for 1 day. The cells were starved with serum-free medium for 1 day prior to treatment with serum-free media containing 0.5 μCi/ml ¹⁴C-thymidine as well as PAR-1 inhibitor and/or thrombin (where applicable). The cells were then incubated at 37° C. for 48 hours prior to reading radioactivity on a beta counter (1450 Microbeta Plus, Liquid Scintillation Counter (Wallac Software, Perkin Elmer) using the program for ¹⁴C counting on 96-well Cytostar-T scintillating microplate (Amersham Pharmacia Biotech, Piscataway, N.J.).

As illustrated in FIG. 16, ¹⁴C-thymidine incorporation reflects that thrombin-induced cell proliferation in glioma cell line U373 is dose-dependent. Moreover, thrombin-induced cell proliferation is inhibited by PAR-1 inhibitor Formula 1 in a dose-dependent manner. Of note, the IC₅₀ of PAR-1 inhibitor Formula 1 was determined to be 760 nM and the Kb to be 8 nM.

Similarly, as illustrated in FIG. 17, ¹⁴C-thymidine incorporation reflects that thrombin-induced cell proliferation in pancreatic cancer cell line MiaPaCa2 is dose-dependent. Of note, the EC₅₀ of thrombin was determined to be 0.014 nM. Likewise, thrombin-induced cell proliferation (at a thrombin concentration of 1 nM) is inhibited by PAR-1 inhibitor Formula 2 in a dose-dependent manner. Of note, the IC₅₀ of PAR-1 inhibitor Formula 2 was determined to be 0.0676 μM.

Calcein AM Assay

Viability of cells was ascertained using the LIVE/DEAD Viability/Cytotoxicity calcein AM assay [Molecular Probes, technical bulletin MP03224] in the pancreatic cell line MiaPaCa2 and the pancreatic cancer cell line Panc1 following treatment with thrombin, or PAR-1 inhibitor in the presence or absence of thrombin (at a concentration of 10 nM). Calcein AM is a cell-permeant nonflouroescent molecule which is cleaved by esterases found within live cells to create the intensely fluorescent calcein. The polyanionic dye calcein is retained within live cells, producing an intense uniform green fluorescence which can be measured by a fluorescent multiwell plate reader to determine the actual number of live cells in each well. In brief, 1×10⁴ cells per well were plated into a 96-well plate and allowed to attach overnight at 37° C. prior to treatment with serum-free media containing 10 nM thrombin as well as the appropriate concentration of PAR-1 inhibitor Formula 2 (5×10⁻⁹; 1×10⁻⁸; 5×10⁻⁸; 1×10⁻⁷; 5×10⁻⁷; 1×10⁻⁶; 5×10⁻⁶; or 1×10⁻⁵ M). Cells were then incubated at 37° C. for 3 days prior to adding Calcein AM reagent and reading fluorescence.

As illustrated in FIG. 18, thrombin-induced cell proliferation in pancreatic cell line MiaPaCa2 is dose-dependent. The thrombin-induced cell proliferation was inhibited by the PAR-1 inhibitor Formula 2. In contrast, thrombin-induced cell proliferation in pancreatic cancer cell line Panc1 was minimal as was its inhibition by the PAR-1 inhibitor Formula 2.

Cell Titerglo Assay

Cell Titerglo Luminescent Cell Viability Assay (Promega, Technical bulletin 288) is a method of determining the number of viable cells in culture based on the quantitation of ATP present that signals metabolically active cells. The amount of ATP is directly proportional to the number of cells present in culture.

In brief, 4×10³ cells per well were plated into a 96-well plate and allowed to attach overnight at 37° C. prior to treatment with serum-free media containing 10 nM thrombin as well as the appropriate concentration of PAR-1 inhibitor Formula 2 (5×10⁻⁹; 1×10⁻⁸; 5×10⁻⁸; 1×10⁻⁷; 5×10⁻⁷; 1×10⁻⁶; 5×10⁻⁶; or 1×10⁻⁵ M). Cells were then incubated at 37° C. for 4 days prior to adding Cell Titerglo reagent and reading luminescence.

As illustrated in FIG. 19, the Cell Titerglo data demonstrates that PAR-1 inhibitor Formula 2 inhibited cell proliferation in multiple cell lines (i.e., the ovarian cancer cell lines SKOV-3; the pancreatic cancer cell line MiaPaCa2; the prostate cancer cell line PC-3; and glioma cell lines U373, SNB19, SF295, and T98G)).

Soft Agar Assay

Cell proliferation was ascertained by soft agar assay. In brief, cells were grown within a matrix of 1.2% soft agar that contained PAR-1 inhibitor at the appropriate concentration. Plates containing cells in the soft agar were incubated at 37° C. for approximately 2 weeks until cell colonies were visible. Cells were then stained by adding 1 mg/ml MTT in PBS and incubating at 37° C. for 1-2 hours. After which the staining solution was removed and plates incubated at 4° C. for 1-2 hours to resolidify agar before scanning.

As illustrated in FIG. 20, cell proliferation in the pancreatic cancer cell line MiaPaCa2 was inhibited in a dose-dependent manner when treated with PAR-1 inhibitor Formula 2 or PAR-1 inhibitor Formula 3. Of note, the IC₅₀ of PAR-1 inhibitor Formula 2 was determined to be 5.7 μM and that of PAR-1 inhibitor Formula 3 was determined to be 5.2 μM.

Similarly, as illustrated in FIG. 21, cell proliferation in the glioma cancer cell line SF295 was inhibited in a dose-dependent manner when treated with PAR-1 inhibitor Formula 2 or PAR-1 inhibitor Formula 3. Of note, the IC₅₀ of PAR-1 inhibitor Formula 2 was determined to be 2.9 μM and that of PAR-1 inhibitor Formula 3 was determined to be 6.1 μM.

Both PAR-1 inhibitor Formula 2 and PAR-1 inhibitor Formula 3 show modest anti-proliferative effects at higher doses in the pancreatic and glioma cancer cell lines MiaPaCa2 and SF295, respectively. Notably, the anti-proliferative effects of the PAR-1 inhibitor Formula 2 was more striking in the soft agar than the Cell Titerglo assay.

Pharmokinetics In Vivo

In preparation for in vivo studies, the pharmokinetics of PAR-1 inhibitor Formula 2 was examined in nude mice. Rising dose levels of PAR-1 inhibitors were administered to mice through various routes (i.e., p.o., i.p., and s.c.). Blood plasma samples were collected at various times after dosing over a period of 24 hours. Quantitation was achieved using high-performance liquid chromatography (HPLC)-atmospheric pressure chemical ionization (APCI) tandem mass spectrometry. Following p.o. administration of PAR-1 inhibitor Formula 2 at a dose of 40 mpk, the mean trough mouse plasma concentration of PAR-1 inhibitor Formula 2 was determined to be 9.9 μM at 8 hours and 0.9 μM at 12 hours.

In Vivo Xenograft Model

PAR-1 inhibitor Formula 2 was evaluated in three human xenograft tumor models, Mia-PaCa2 (pancreatic cancer), U373 MG (glioma), MDA-MB-231 (breast cancer).

In brief, for each xenograft model, 5×10⁶ cells from a tumor cell line were inoculated into a mouse subcutaneously to induce tumor growth. Athymic nude mice were used for xenografts of MiaPaCa2 and U373 MG tumor cells, whereas SCID (severe combined immunodeficient) mice were used for xenografts of MDA-MB-231 cells. Treatment commenced when tumor size averaged 50 mm³. The day before treatment started, tumors were measured and tumor bearing mice were randomized into treatment groups (10 mice each group). These treatment groups included 20% HP-β-CD as a vehicle control, a cytotoxic agent as a positive control, and increasing dose levels of PAR-1 inhibitor Formula 2. Treatment with vehicle control or PAR-1 inhibitor Formula 2 was administered by twice daily oral gavage. For the U373 model, treatment with PAR-1 inhibitor Formula 2 was administered twice a day at dose levels of 15, 30, and 60 mpk; for the positive control, 16 mpk temzolomide was injected intraperitoneally every other day. For the Mia-PaCa2 model, treatment with PAR-1 inhibitor Formula 2 was administered twice a day at dose levels of 20, 40, and 60 mpk; for the positive control, 150 mpk gemcitabine was administered intraperitoneally twice a week. For the MDA-MB-231 model, treatment with PAR-1 inhibitor Formula 2 was administered twice a day at dose levels of 5, 10, and 20 mpk; for the positive control, 100 mpk cyclophosphamide (Cytoxan) was injected intraperitoneally twice a week. Tumor size and body weight were measured twice a week. On the last day, blood and tumor samples were collected at 1 hour, 3 hours, and 6 hours after the final dose was administered.

In Vivo Breast Cancer Lung Metastasis Model

MDA-MB-231 tumor cells from xenografts in SCID mice naturally metastasize from the primary tumor site to the lung of the host animal. For this model, in addition to the change in growth of primary tumors, the number of tumor cell colonies metastasized to the lungs was also quantified. In brief, after animals were euthanized on the last day of the study, lungs were removed from each animal and preserved in 10% formaldehyde solution. Preserved lungs were then processed through paraffin embedding, microtome thin-sectioning, and hematoxylin/eosin staining. Sections of lungs were then examined under an optical microscope at 10×40 magnification. Tumor colonies in each of 10 optical fields of each section were enumerated and rated according to an index as following: 0=no tumor colony found; 1=colonies occupying 1-10% of the field; 2=colonies occupying 11-20% of the field; 3=colonies occupying 21-30% of the field; 4=colonies occupying 31-40% of the field; 5=colonies occupying 41-50% of the field; 6=colonies occupying 51-60% of the field. For each section slide, rating of each of 10 fields were averaged.

As illustrated in FIG. 27, lung metastasis in xenograft tumors of breast cancer cell line MDA-MB-231 decreased following treatment with PAR-1 inhibitor Formula 2 at a dose of 20 mpk.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed:

1. A method for preventing and/or treating the growth and/or metastasis of a cell proliferative disorder in a patient disposed to or suffering therefrom comprising administering a therapeutically effective amount of a protease-activated receptor-1 (PAR-1) inhibitor.

2. The method of claim 1, wherein the cell proliferative disorder is renal cancer.

3. The method of claim 1, wherein the cell proliferative disorder is hepatocellular carcinoma.

4. The method of claim 1, wherein the cell proliferative disorder is pancreatic cancer.

5. The method of claim 1, wherein the cell proliferative disorder is a glioma.

6. The method of claim 5, wherein the glioma is an anaplastic astrocytoma.

7. The method of claim 5, wherein the glioma is a glioblastoma multiforme.

8. The method of claim 1, wherein the PAR-1 inhibitor is: 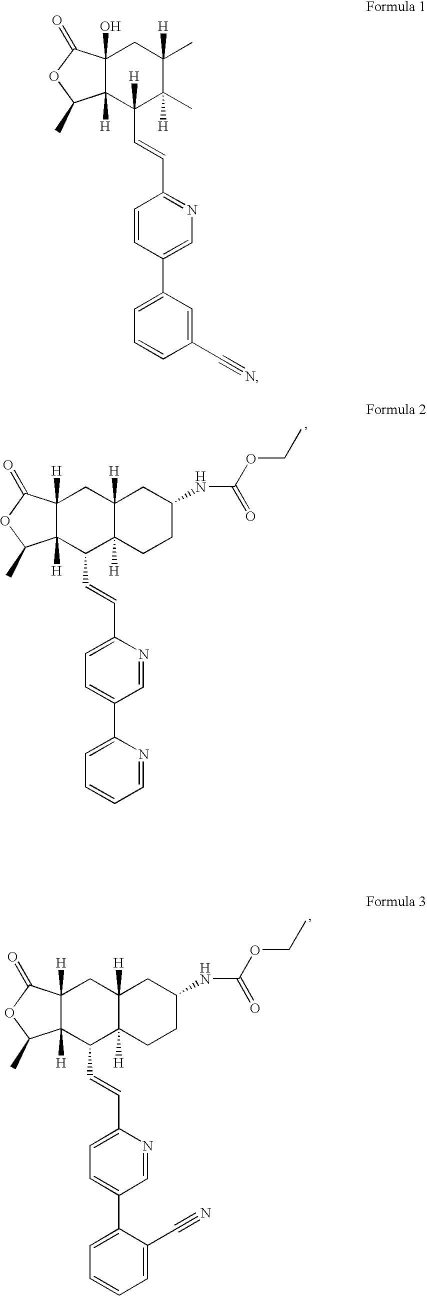 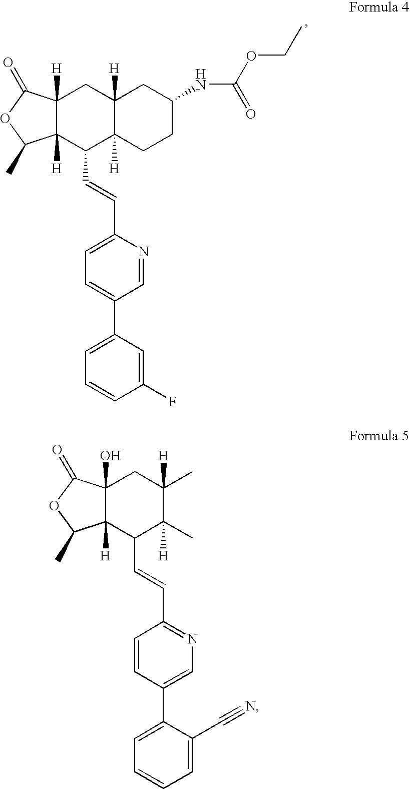
BMS-200261, RWJ-56110, RWJ-58259, a blocking antibody to PAR-1, a pepducin to PAR-1, an antisense oligonucleotide to the nucleic acid encoding PAR-1, a small interfering RNA or a short hairpin RNA to the mRNA encoding PAR-1, a pharmaceutically acceptable salt of any of the above, or a combination of two or more of the above.

9. The method of claim 1, wherein the PAR-1 inhibitor is: 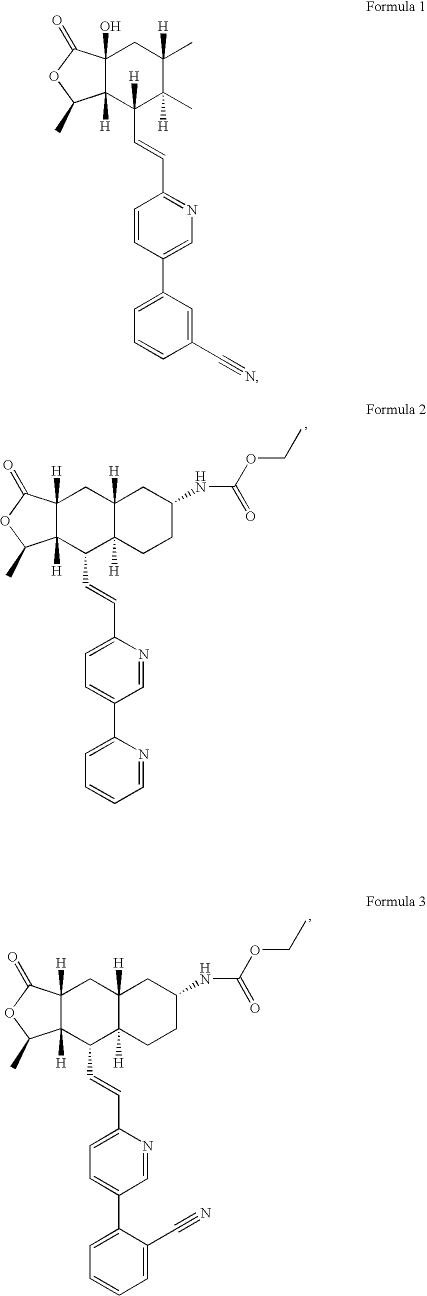 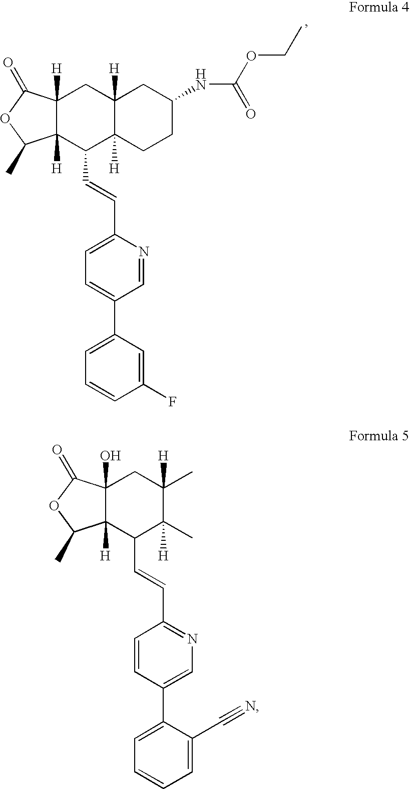
a pharmaceutically acceptable salt of any of the above, or a combination of two or more of the above.

10. The method of claim 1, wherein the PAR-1 inhibitor is Formula 1 or a pharmaceutically acceptable salt thereof.

11. The method of claim 1, wherein the PAR-1 inhibitor is Formula 2 or a pharmaceutically acceptable salt thereof.

12. The method of claim 1, wherein the PAR-1 inhibitor is Formula 3 or a pharmaceutically acceptable salt thereof.

13. The method of claim 1, wherein the PAR-1 inhibitor is administered in an amount sufficient to maintain the patient's plasma level of the PAR-1 inhibitor at or above 1 μM for 24 hrs.

14. The method of claim 1 further comprising administering an other antineoplastic agent.

15. The method of claim 14, wherein the other antineoplastic agent is temozolomide and the cell proliferative disorder is a glioma.

16. The method of claim 14, wherein the other antineoplastic agent is interferon and the cell proliferative disorder is melanoma.

17. The method of claim 14, wherein the other antineoplastic agent is PEG-Intron (peginterferon alpha-2b) and the cell proliferative disorder is melanoma.
